(19) United States
(12) Patent Application Publication
(10) Pub. No.: US 2002/0019993 A1
(43) Pub. Date: Feb. 14, 2002

(54) Full term development of animals from enucleated oocytes reconstituted with adult somatic cell nuclei

(76) Inventors: Teruhiko Wakayama, Honolulu, HI (US); Ryuzo Yanagimachi, Honolulu, HI (US)

Correspondence Address: Barbara E. Arndt, Ph.D., JONES, DAY, REAVIS & POGUE, 901 Lakeside Avenue, Cleveland, OH, 44114, US

(21) Appl. No.: 09/919,106

(22) Filed: Jul. 31, 2001

Related U.S. Application Data
(63) Continuation of application No. 09/233,923, filed on Jan. 20, 1999.
Application No. 09/233,923 is a continuation-in-part of application No. 09/132,104, filed on Aug. 10, 1998.
(60) Provisional application No. 60/072,002, filed on Jan. 21, 1998.
(60) Provisional application No. 60/089,940, filed on Jun. 19, 1998.

(51) Int. Cl. A01K 67/027

(52) U.S. Cl. 800/21; 800/18; 800/14; 800/19; 800/20; 800/8

(57) ABSTRACT

Animals are produced following injection of adult cumulus or fibroblast cell nuclei into enucleated oocytes. The invention provides a method for cloning an animal by directly inserting an adult somatic cell nucleus into a recipient enucleated oocyte. Preferably, the nucleus is inserted by microinjection and, more preferably, by piezo electrically-actuated microinjection. The oocyte is activated prior to, during, or up to about 6 hours after insertion of the nucleus, by electroactivation or exposure to a chemical activating agent, such as Sr²⁺. The activated renucleated oocyte is allowed to develop into an embryo and is transplanted to a host surrogate mother to develop into a live offspring.

This application is a continuation-in-part of U.S. patent application Ser. No. 09/132,104, filed Aug. 10, 1998, which claims the benefit of U.S. Provisional Patent Applications, Ser. No. 60/072,002, filed Jan. 21, 1998, and Ser. No. 60/089,940, filed Jun. 19,1998.

The U.S. Government has a paid-up license in this invention and the right in limited circumstances to require the patent owner to license others on reasonable terms as provided for by the terms of contract No. R01-HD-03402 awarded by the National Institutes of Health, Public Health Service.

### BACKGROUND OF THE INVENTION

The invention relates to the cloning of animals by the insertion of a nucleus of an adult somatic cell into an enucleated oocyte in such a way that the host oocyte forms an embryo and can develop into a live animal. In one embodiment of the invention, insertion of a nucleus is accomplished by piezo electrically-actuated microinjection.

The rapid production of large numbers of near-identical animals is very desirable. For example, it is expected that broad medical benefits may be obtained when the near-identical animals are also genetically engineered (e.g., transgenic) animals. Genetically altered large animals can act as living pharmaceutical “factories” by producing valuable pharmaceutical agents in their milk or other fluids or tissue (a production method sometimes referred to as “pharming”) or act as living organ or cell “factories” for human organs or cells that will not be rejected by the human immune system. The production of large numbers of near-identical research animals, such as mice, guinea pigs, rats, and hamsters is also desirable. For example, the mouse is a primary research model for the study of mammalian biology, and the availability of near-identical, transgenic or non-transgenic, mice would be very beneficial in the analysis of, for example, embryonic development, human diseases, and for testing of new pharmaceuticals. Thus, for a variety of reasons, (e.g., in the context of breeding farm animals, or the interpretation of data generated in mice), it may be desirable to reliably produce offspring of a particular animal that are genetically near-identical to the parent.

Further, with respect to transgenesis, current protocols for generating transgenic animals are not sufficiently advanced to guarantee the programmed control of gene expression in the context of the whole animal. Although it is possible to minimize detrimental “position” effects caused by the quasi-random manner in which the transgene integrates into the host genome, differences can exist in transgene expression levels between individuals carrying the same transgene construct inserted at the same locus in the same copy number. Thus, generating even modest numbers of transgenic animals producing the desired levels of any given recombinant protein(s) can be very time-consuming and expensive. These problems may be exacerbated because the number of transgenic offspring is often low (commonly only one) due to low efficiency, and many transgenic founders are infertile.

One approach to solving these problems is to “clone” genetically near-identical animals from the cells of transgenic or non-transgenic adult animals that have a desired trait or produce a target product at the desired level. To this end, colonies of genetically near-identical animals (clones) could be generated relatively rapidly from the cells of a single adult animal. Moreover, selective and reliable cloning of adult animals that produce increased yields of milk and meat could rapidly produce large numbers of high producers. Cloning of animals from adult somatic cells could also be beneficial in the reproduction of pets (e.g., dogs, cats, horses, birds, etc.) and rare or endangered species. As used herein, “cloning” refers to the full development to adulthood of an animal whose non-mitochondrial DNA may be derived from a somatic donor cell through the transfer of nuclear chromosomes from the somatic donor cell to a recipient cell (such as an oocyte) from which the resident chromosomes have been removed.

In normal mammalian development, oocytes become developmentally arrested at the germinal vesicle (GV) stage in prophase of the first meiotic division. Upon appropriate stimulation (e.g., a surge in plasma luteinizing hormone), meiosis resumes, the germinal vesicle breaks down, the first meiotic division is completed and the oocyte then becomes arrested at metaphase of the second meiosis (“Met II”). Met II oocytes can then be ovulated and fertilized. Once fertilized, the oocyte completes meiosis with the extrusion of the second polar body and the formation of male and female pronuclei. The embryos begin to develop by undergoing a series of mitotic divisions before differentiating into specific cells, resulting in the organization of tissues and organs. This developmental program ensures the successful transition from oocyte to offspring.

Although the cells of early embryos have classically been regarded as totipotent (that is, that they are capable of developing into a new individual per se), this totipotency is lost following a small number of divisions, that number varying between species (e.g., murine and bovine embryos). The mechanisms underlying this apparent loss of totipotency are poorly understood but are presumed to reflect subtle changes in the DNA environment affecting gene expression, that are collectively termed “reprogramming”. Without being bound by theory, it is believed that cloning techniques could possibly either subvert or mimic “reprogramming”.

Given the enormous practical benefits of cloning, there has been a commensurately great interest in overcoming technological barriers and developing new techniques for the fusion of either embryonic cells or fetal cells with enucleated oocytes. To date, however, there has been a lack of reported protocols that have reproducibly generated full term development of clones from adult somatic cells. For example, it has been reported that when bovine cumulus cell nuclei were injected into enucleated oocytes which were then electroactivated, 9% of 351 injected oocytes developed to blastocysts, but none developed to term. Likewise, Sendai virus-mediated fusion of adult mouse thymocytes with enucleated Met II oocytes, followed by activation thirty to sixty minutes later with 7% ethanol, resulted in 75% of 20 oocytes reaching the 2-cell stage, but none developed beyond the 4-cell stage.

A recent report describes the electrofusion of cultured “mammary gland cells” with enucleated oocytes to produce a single live offspring sheep, which was named “Dolly” (Wilmut, I. et al. (1997), Nature 385, 810-813). Dolly is reported to have developed from one of 434 enucleated oocytes electrofused with cells derived from the mammary gland that had been cultured for five days under conditions of serum starvation. According to the method reported to have been used to clone Dolly, the “mammary gland cell” was inserted by micropipette into the perivitelline space of an enucleated oocyte. Wilmut reports that the cells were immediately subjected to an electric pulse to induce membrane fusion and activate the oocyte to trigger resumption of the cell cycle. The resulting embryo (in addition to 28 others in the experiment) was transferred into a suitable recipient and, in this single case, the pregnancy proceeded to produce Dolly. However, because the “mammary gland cell” was from a cell line established from a 6-year old sheep that was in the third trimester of pregnancy, doubt has been publicly expressed as to the identity of the cells from which the donor nucleus was obtained, and even whether that cell was of adult origin.

In our co-owned, copending U.S. patent application Ser. No. 09/132,104, of which the present application is a continuation-in-part, we disclosed and claimed a controllable and efficient method of cloning animals from adult somatic cells, as exemplified by the successful production of cloned fertile mice from adult cumulus cell nuclei. We also disclosed that the method could be successfully used to produce clones of the cloned mice. Since the source of the donor cumulus cells is female, all the cloned mice produced were female.

### SUMMARY OF THE INVENTION

The present invention is an extension of the method of the invention to include the successful production of cloned, live offspring from fibroblast cells from adult animals. In particular, the method of the invention provides cloned, live offspring from fibroblasts from adult male animals, showing that the invention method is not limited to producing female cloned animals. In an embodiment of the invention, the fibroblast cells are cultured for a period of time prior to their use as nuclear donors to produce cloned animals.

The method of the invention for cloning animals from adult somatic cells includes the steps of inserting the nucleus of the somatic cell (or a portion of the nuclear contents including at least the minimum chromosomal material able to support development) into the cytoplasm of an enucleated oocyte, and facilitating embryonic development of the reconstituted oocyte to result in a live offspring. As used herein, the term “adult somatic cell” means a cell from a post-natal animal, which is therefore neither a fetal cell nor an embryonic cell, and which is not of the gamete lineage. The resulting viable offspring is a clone of the animal that originally provided the somatic cell nucleus for injection into the oocyte. The invention is applicable to cloning of all animals, including amphibians, fish, birds (e.g., domestic chickens, turkeys, geese, and the like) and mammals, such as primates, ovines, bovines, porcines, ursines, felines, canines, equines, rodents, and the like.

In one embodiment of the invention, the donor adult somatic cell is “2n”; that is, it possesses the diploid complement of chromosomes as seen in G0 or G1 of the cell cycle. The donor cell may be obtained from an in vivo source or may be from a cultured cell line. An example of an in vivo source of the 2n donor nucleus (i.e., in G0 or G1 phase) is a cumulus cell. Cumulus (Latin for “a little mound”) cells are so-called because they form a solid mass (heap) of follicular cells surrounding the developing ovum prior to ovulating. Following ovulation in some species, such as mice, many of these cells remain associated with the oocyte (to form the cumulus oophorus) and, in mice, more than 90% are in G0/G1 and, therefore, are 2n. The invention contemplates using donor nuclei taken from other in vivo or in vitro (i.e., cultured) sources of 2n adult somatic cells including, without limitation, epithelial cells, neural cells, epidermal cells, keratinocytes, hematopoietic cells, melanocytes, chondrocytes, lymphocytes, macrophages, monocytes, nucleated erythrocytes, fibroblasts, Sertoli cells, cardiac muscle cells, skeletal muscle cells, smooth muscle cells, and other cells from organs including, without limitation, skin, lung, pancreas, liver, kidney, urinary bladder, stomach, intestine, bone, and the like, and their progenitor cells where appropriate.

In another embodiment of the invention, the donor adult somatic cell is “2C to 4C”; that is, it contains one to two times the diploid genomic content, as a result of replication during S phase of the cell cycle. This donor cell may be obtained from an in vivo or an in vitro source of actively dividing cells including, but not limited to, epithelial cells, hematopoietic cells, epidermal cells, keratinocytes, fibroblasts, and the like, and their progenitor cells where appropriate.

An embodiment of the method of the invention includes the steps of (i) allowing the nucleus (or portion thereof including the chromosomes) to be in contact with the cytoplasm of the enucleated oocyte for a period of time (e.g., up to about 6 hours) after insertion into the oocyte, but prior to activation of the oocyte, and (ii) activating the oocyte. In this embodiment, the nucleus is inserted into the cytoplasm of the enucleated oocyte by a method that does not concomitantly activate the oocyte.

When a donor nucleus having 2n chromosomes is employed, the method further includes the step of disrupting microtubule and/or microfilament assembly for the period of time after insertion of the nucleus into the enucleated oocyte in order to suppress the formation of a polar body and maintain the 2n chromosome number. When, for example, a 4n donor nucleus is employed, this step of the method is omitted such that a polar body is formed, and the ploidy of the renucleated oocyte can be reduced to 2n.

In a preferred embodiment of the invention, the nucleus is inserted by microinjection and, more preferably, by piezo electrically-actuated microinjection. The use of a piezo electric micromanipulator enables harvesting and injection of the donor nucleus to be performed with a single needle. Moreover, the enucleation of the oocyte and injection of the donor cell nucleus can be performed quickly and efficiently and, consequently, with less trauma to the oocyte than with previously reported methods, such as the fusing of the donor cell and oocyte mediated by fusion-promoting chemicals, by electricity or by a fusogenic virus.

The introduction of nuclear material by microinjection is distinct from cell fusion, temporally and topologically. By the microinjection method, the plasma membrane of the donor cell is punctured (to enable extraction of the nucleus) in one or more steps that are temporally separated from delivery of that nucleus (or a portion thereof including at least the chromosomes) into an enucleated oocyte, also following plasma membrane puncture. Separate puncturing events are not a feature of cell fusion.

Furthermore, the spatiotemporal separation of nucleus removal and introduction allows controlled introduction of materials in addition to the nucleus. The facility to remove extraneous cytoplasm and to introduce additional materials or reagents may be highly desirable. For example the additive(s) may advantageously modulate the embryological development of the renucleated oocyte. Such a reagent may comprise an antibody, a pharmacological signal transduction inhibitor, or combinations thereof, wherein the antibody and/or the inhibitor are directed against and/or inhibit the action of proteins or other molecules that have a negative regulatory role in cell division or embryonic development. The reagent may include a nucleic acid sequence, such as a recombinant plasmid or a transforming vector construct, that may be expressed during development of the embryo to encode proteins that have a potential positive effect on development and/or a nucleic acid sequence that becomes integrated into the genome of the cell to form a transformed cell and a genetically altered animal. The introduction of a reagent into a cell may take place prior to, during, or after the combining of a nucleus with an enucleated oocyte.

### BRIEF DESCRIPTION OF THE FIGURES

The file of this patent contains at least one drawing executed in color. Copies of this patent with color drawing(s) will be provided by the Patent and Trademark Office upon request and payment of the necessary fee.

FIG. 1A is a photomicrograph of a live ovulated oocyte surrounded by cumulus cells. The egg coat, the zona pellucida, appears as a relatively clear zone around the oocyte.

FIG. 1B is a photomicrograph taken within 10 minutes after injection of a cumulus cell nucleus into the cytoplasm of an enucleated oocyte, showing the intact cumulus cell nucleus within the oocyte cytoplasm. Oocytes injected with cumulus cell nuclei were fixed, stained and photographed with a phase contrast microscope.

FIG. 1C is a photomicrograph showing transformation of the nucleus into apparently disarrayed chromosomes 3 hours after injection of the nucleus. The disarray reflects an unusual situation in which single, condensed chromatids are each attached to a single pole of the spindle, and are therefore not aligned on a metaphase plate.

FIG. 1D is a photomicrograph taken 1 hour after activation of the oocyte with Sr²⁺ showing chromosomes segregated into two groups. (mb=midbody).

FIGS. 1E and 1E′ are photomicrographs taken 5 hours after activation of the oocyte with Sr²⁺ showing two pseudo-pronuclei with a varying number of distinct nucleolus-like structures discernable per egg. The size and number of pseudo-pronuclei varied, suggesting ‘random’ segregation of chromosomes following oocyte activation.

FIG. 1F is a photomicrograph of live blastocysts produced following injection of enucleated oocytes with cumulus cell nuclei.

FIG. 2A is a photograph of four-week-old (cloned mouse) Cumulina (foreground) with her foster mother.

FIG. 2B is a photograph of Cumulina at 2.5 months with the pups she produced following mating with a CD-1(albino) male.

FIG. 2C is a photograph of two B6C3F1-derived, cloned, agouti young (center) in front of their albino foster mother (CD-1), a B6D2F1 oocyte donor (black, right), and the B6C3F1 cumulus cell donor (agouti, left). The two agouti offspring in the center are clones (identical ‘twin’ sisters) of the agouti cumulus cell donor and are two of the offspring described in Series C (see text) and Table 2.

FIG. 3 illustrates the development following uterine transfer of embryos derived following injection of Sertoli cell nuclei into enucleated oocytes. FIG. 3A is a photomicrograph of the uteri of recipient females 8.5 days post coitum (dpc), fixed with Bouin's fluid, dehydrated and cleared with benzyl benzoate. All uterine implantation sites failed to develop except in one (arrow) where an embryo (FIG. 3b) appeared normal and was in the circa 12 somite stage.

FIG. 4 represents DNA typing of donors and offspring in Series C (see text and Table 2) that corroborates genetic identity between cloned offspring and cumulus cell donors, and non-identity between oocyte donors and host foster females. Placental DNA from the six cloned Series C offspring (lanes 10-15) was compared with DNA from the three cumulus cell donor females (lanes 1-3), the three oocyte recipient females (lanes 4-6), and the three host females (lanes 7-9). Control DNA was from C57BL/6 (lane 16), C3H (lane 17), DBA/2 (lane 18), B6C3F1 (lane 19) and B6D2F1 (lane 20). 100 base pair (bp) DNA size marker ladders are shown on the left of FIGS. 4A and 4B. FIG. 4A illustrates PCR typing using the strain-specific marker D1Mit46. FIG. 4B illustrates PCR typing using the strain-specific marker D2Mit102. PCR-amplified DNA (FIG. 4A and FIG. 4B) from F1 hybrid mice exhibit an additional gel band not seen in DNA from the inbred parental strains (lanes 16-20). This extra band corresponds to a heteroduplex derived from the two parental products, whose conformation results in anomalous gel migration. FIG. 4C illustrates Southern blot typing of strain-specific Emv loci (Emv1, Emv2 and Emv3).

FIG. 5 is a schematic representation of the cloning procedure of the present invention.

### DETAILED DESCRIPTION OF THE INVENTION

The mitotic cell cycle ensures that every cell that divides donates equal genetic material to two daughter cells. DNA synthesis does not occur throughout the cell division cycle but is restricted to a part of it, namely the synthetic phase (or “S” phase) before mitosis. A gap of time (G2) occurs after DNA synthesis and before cell division; another gap (G1) occurs after division and before the next S phase. The cell cycle thus consists of the M (mitotic) phase, a G1 phase (the first gap), the S phase, a G2 phase (the second gap), and back to M. Many nondividing cells in tissues (for example, all resting fibroblasts) suspend the cycle after mitosis prior to S phase. Such “resting” cells which have exited from the cell cycle before S phase, are said to be in the G0 state. Cells entering G0 can remain in this state temporarily or for very long periods. Sertoli cells and neurons, for example, characteristically do not divide in adult animals but remain at G0. More than 90% of cumulus cells surrounding recently ovulated (mouse) oocytes are in G0 or G1. The nuclei of cells in G0 or G1 have a diploid (2n) DNA content, i.e., they have two copies of each morphologically distinct chromosome (of n−1 autosomal chromosome types). The nuclei of cells in G2 have a 4C DNA content, i.e., during S phase, DNA in each of the two copies of the each of the distinct chromosomes has been replicated.

The present invention describes a method for generating clones of vertebrate animals. In the method, each clone develops from an enucleated oocyte that has received the nucleus (or a portion thereof including, at least, the chromosomes) of an adult somatic cell. In one embodiment of the invention, cloned mice were born following microinjection of the nuclei of cumulus cells (i.e., ovulated ovarian follicle cells) into enucleated oocytes by the method of the invention. In another embodiment of the invention, cloned mice were born following microinjection of the nuclei of adult tail fibroblasts into enucleated oocytes by the method of the invention. In embodiments of the invention employing fibroblasts, some fibroblasts were cultured in vitro in media that did not contain serum; thus, these fibroblasts were “starved” in order to induce them to remain in G0 or G1 phase of the cell cycle, as known to those skilled in the art, and they are presumed to contain 2n chromosomes. Other fibroblasts were cultured in vitro in media that contained serum; thus, these fibroblasts continued the cell cycle through division and were presumed to be 2C to 4C. In further embodiments of the invention, thymus cells, spleen cells, macrophages were used as the adult somatic cell nuclear donors.

Additional animals such as, but not limited to, primates, cattle, pigs, cats, dogs, horses, and the like, may be also cloned by the method of the invention. The invention method is shown herein to provide a high rate of successful development of embryos to the morula/blastocyst stage, a high rate of implantation of transferred embryos in recipient foster mammals, and a greater than 2% success rate of resulting newborn mammals. The magnitude of these efficiencies means that the method of the invention is readily reproducible.

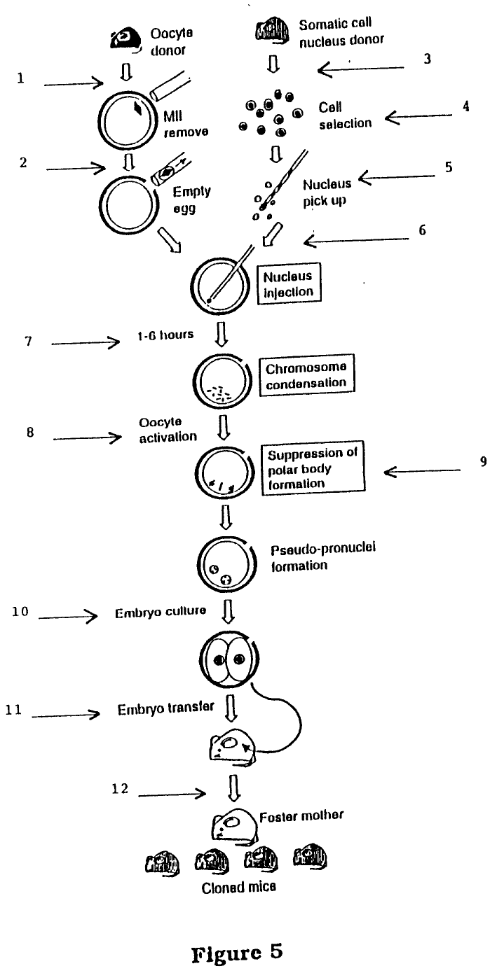

Steps and substeps of one embodiment of the method of the invention for cloning an animal are illustrated in the example of FIG. 5. Briefly, oocytes are harvested (1) from an oocyte donor animal and the Met II plate is removed (2) to form an enucleated oocyte (chromosomally “empty” egg). Somatic cells are harvested (3) from an adult donor, healthy-looking cells are selected (4), and their nuclei (or nuclear constituents including the chromosomes) are obtained (5). A single nucleus is injected (6) into the cytoplasm of an enucleated oocyte. The nucleus is allowed to reside within the cytoplasm of the enucleated oocyte (7) for up to 6 hours, during which time chromosome condensation may be observed. The oocyte is then activated (8) in the presence of an inhibitor of microtubule and/or microfilament assembly (9) to suppress the formation of a polar body. During this activation time period, the formation of pseudo-pronuclei may be observed. Eggs forming pseudo-pronuclei are selected and placed in embryo culture (10). The embryos are then transferred (11) to foster surrogate mothers, to permit the birth (12) of live offspring.

Thus, one embodiment of the method of the invention for cloning a mammal comprises the following steps: (a) collecting a somatic cell nucleus, or a portion thereof containing at least the chromosomes, from a somatic cell of an adult mammal; (b) inserting the at least that portion of the somatic cell nucleus into an enucleated oocyte to form a renucleated oocyte; (c) allowing the renucleated oocyte to develop into an embryo; and (d) allowing the embryo to develop into a live offspring. Each of these steps is described below in detail. The somatic cell nucleus (or nuclear constituents containing the chromosomes) may be collected from a somatic cell that has greater than 2n chromosomes (e.g., one which has one to two times the normal diploid genomic content). Preferably, the somatic cell nucleus is collected from a somatic cell that has 2n chromosomes. Preferably, the somatic cell nucleus is inserted into the cytoplasm of the enucleated oocyte. The insertion of the nucleus is preferably accomplished by microinjection and, more preferably, by piezo electrically-actuated microinjection.

Activation of the oocyte may take place prior to, during, or after the insertion of the somatic cell nucleus. In one embodiment, the activation step takes place from zero to about six hours after insertion of the somatic cell nucleus in order to allow the nucleus to be in contact with the cytoplasm of the oocyte for a period of time prior to activation of the oocyte. Activation may be achieved by various means including, but not limited to, electroactivation, or exposure to ethyl alcohol, sperm cytoplasmic factors, oocyte receptor ligand peptide mimetics, pharmacological stimulators of Ca²⁺ release (e.g., caffeine), Ca²⁺ ionophores (e.g., A2318, ionomycin), modulators of phosphoprotein signaling, inhibitors of protein synthesis, and the like, or combinations thereof. In one embodiment of the invention, the activation is achieved by exposing the cell to strontium ions (Sr²⁺).

Activated, renucleated oocytes injected with 2n chromosomes are preferably exposed to a microtubule and/or microfilament disrupting agent (described below) to prevent the formation of a polar body, thus retaining all the chromosomes of the donor nucleus within the renucleated host oocyte. Activated, renucleated oocytes injected with 2C to 4C nuclei are preferably not exposed to such an agent, in order to allow the formation of a polar body to reduce the number of chromosomes to 2n.

The step of allowing the embryo to develop may include the substep of transferring the embryo to a female mammalian surrogate recipient, wherein the embryo develops into a viable fetus. The embryo may be transferred at any stage, from two-cell to morula/blastocyst stage, as known to those skilled in the art.

Embodiments of the present invention may have one or more of the following advantages, as well as other advantages not listed. First, nucleus delivery (or delivery of nuclear constituents including the chromosomes) by microinjection is applicable to a wide variety of cell types—whether grown in vitro or in vivo—irrespective of size, morphology, developmental stage of donor, and the like. Second, nucleus delivery by microinjection enables careful control (e.g., minimization) of the volume of nucleus donor cell cytoplasm and nucleoplasm introduced into the enucleated oocyte at the time of nuclear injection, as extraneous material may “poison” developmental potential. Third, nucleus delivery by microinjection allows carefully controlled co-injection (with the donor nucleus) of additional agents into the oocyte at the time of nuclear injection. These are exemplified below. Fourth, nucleus delivery by microinjection allows a period of exposure of the donor nucleus to the cytoplasm of the enucleated oocyte prior to activation. This exposure may allow chromatin remodeling/reprogramming which favors subsequent embryonic development. Fifth, nucleus delivery by microinjection allows a wide range of choices for subsequent activation protocol (in one embodiment, the use of Sr²⁺). Different activation protocols may exert different effects on developmental potential. Sixth, activation may be in the presence of microtubule- and/or microfilament disrupting agents (in one embodiment, cytochalasin B) to prevent chromosome extrusion, and modifiers of cellular differentiation (in one embodiment, dimethylsulfoxide) to promote favorable developmental outcome. Seventh, in one embodiment, nucleus delivery is by piezo electrically-actuated microinjection, allowing rapid and efficient processing of samples and thereby reducing trauma to cells undergoing manipulation. This is, in part, because somatic nucleus preparation and introduction into the enucleated oocyte may be performed with the same injection needle (in contrast to conventional microinjection protocols which require at least one change of injection needle between coring of the zona pellucida and puncturing of the oocyte plasma membrane). Moreover, the oocytes of some species (e.g., mouse) are not amenable to microinjection using conventional needles, whereas piezo electrically-actuated microinjection affords a high success rate. Finally, not only individual steps in the present invention, but their relationship to each other as a whole, results in a high cloning efficiency. The individual steps are now presented in greater detail to show how they are arranged in respect of one to the other in the present invention.

The Recipient Oocytes.

The stage of in vivo maturation of the oocyte at enucleation and nuclear transfer has been reported to be significant to the success of nuclear transfer methods. In general, reports of mammalian nuclear transfer describe the use of Met II oocytes as recipients. Met II oocytes are of the type normally activated by fertilizing spermatozoa. It is known that the chemistry of the oocyte cytoplasm changes throughout the maturation process. For example, a cytoplasmic activity associated with maturation, metaphase-promoting factor (“MPF”), is maximal in immature oocytes at metaphase of the first meiotic division (“Met I”), declining with the formation and expulsion of the first polar body (“Pb1”), and again reaching high levels at Met II. MPF activity remains high in oocytes arrested at Met II, rapidly diminishing upon oocyte activation. When a somatic cell nucleus is injected into the cytoplasm of a Met II oocyte (i.e., one with high MPF activity), its nuclear envelope breaks down and chromatin condenses, resulting in the formation of metaphase chromosomes.

Oocytes that may be used in the method of the invention include both immature (e.g., GV stage) and mature (i.e., Met II stage) oocytes. Mature oocytes may be obtained, for example, by inducing an animal to super-ovulate by injections of gonadotrophic or other hormones (for example, sequential administration of equine and human chorionic gonadotrophins) and surgical harvesting of ova shortly after ovulation (e.g., 80-84 hours after the onset of estrous in the domestic cat, 72-96 hours after the onset of estrous in the cow and 13-15 hours after the onset of estrous in the mouse). Where it is only possible to obtain immature oocytes, they are cultured in a maturation-promoting medium until they have progressed to Met II; this is known as in vitro maturation (“IVM”). Methods for IVM of immature bovine oocytes are described in WO 98/07841, and for immature mouse oocytes in Eppig & Telfer (Methods in Enzymology 225, 77-84, Academic Press, 1993).

Oocyte Enucleation

Preferably, the oocyte is exposed to a medium containing a microtubule and/or microfilament disrupting agent or actin depolymerizing agent prior to and during enucleation. Disruption of the microfilaments imparts relative fluidity to the cell membrane and underlying cortical cytoplasm, such that a portion of the oocyte enclosed within a membrane can easily be aspirated into a pipette with minimal damage to cellular structures. One microtubule-disrupting agent of choice is cytochalasin B (5 μg/mL). Other suitable microtubule-disrupting agents, such as nocodazole, 6-dimethylaminopurine and colchicine, are known to those skilled in the art. Microfilament depolymerizing agents are also known and include, but are not limited to, cytochalasin D, jasplakinolide, latrunculin A, and the like.

In one preferred embodiment of the invention, the enucleation of the Met II oocyte is achieved by aspiration using a piezo electrically-actuated micropipette. Throughout the enucleation microsurgery, the Met II oocyte is anchored by a conventional holding pipette and the flat tip of a piezo electrically-driven enucleation pipette (internal diameter≈7 μm) is brought into contact with the zona pellucida. A suitable piezo electric driving unit is sold under the name of Piezo Micromanipulator/Piezo Impact Drive Unit by Prime Tech Ltd. (Tsukuba, Ibaraki-ken, Japan). The unit utilizes the piezo electric effect to advance, in a highly controlled, rapid manner, the (injection) pipette holder a very short distance (approximately 0.5 μm). The intensity and interval between each pulse can be varied and are regulated by a control unit. Piezo pulses (for example, intensity=1-5, speed=4-16) are applied to advance (or drill) the pipette through the zona pellucida while maintaining a small negative pressure within the pipette. In this way, the tip of the pipette rapidly passes through the zona pellucida and is thus advanced to a position adjacent to the Met II plate (discernible as a translucent region in the cytoplasm of the Met II oocytes of several species, often lying near the first polar body). Oocyte cytoplasm containing the metaphase plate (which contains the chromosome-spindle complex) is then gently and briskly sucked into the injection pipette in a minimal volume and the injection pipette (now containing the Met II chromosomes) withdrawn slightly. The effect of this procedure is to cause a pinching off of that part of the oocyte cytoplasm containing the Met II chromosomes. The injection pipette is then pulled clear of the zona pellucida, and the chromosomes are discharged into neighboring medium in preparation for microsurgical removal of chromosomes from the next oocyte. Where appropriate, batches of oocytes may be screened to confirm complete enucleation. For oocytes with granular cytoplasm (such as porcine, ovine and feline oocytes), staining with a DNA-specific fluorochrome (e.g., Hoeschst 33342) and brief examination with low UV illumination (enhanced by an image intensified video monitor) is advantageous in determining the efficiency of enucleation.

Enucleation of the Met II oocyte may be achieved by other methods, such as that described in U.S. Pat. No. 4,994,384. For example, enucleation may be accomplished microsurgically using a conventional micropipette, as opposed to a piezo electrically-driven micropipette. This can be achieved by slitting the zona pellucida of the oocyte with a glass needle along 10-20% of its circumference close to the position of the Met II chromosomes (the spindle is located in the cortex of the oocyte by differential interference microscopy). The oocyte is placed in a small drop of medium containing cytochalasin B in a micromanipulation chamber. Chromosomes are removed with an enucleation pipette having an unsharpened, beveled tip.

After enucleation, the oocytes are ready to be reconstituted with adult somatic cell nuclei. It is preferred to prepare enucleated oocytes within about 2 hours of donor nucleus insertion.

Preparation of Adult Somatic Cell Nuclei

Cells derived from populations grown in vivo or in vitro and containing cells with 2n chromosomes (e.g., those in G0 or G1) or greater than 2C chromosomes (e.g., those in G2, which are normally 4C) may be suitable nuclear donors. In one embodiment of the invention, the cells are follicle (cumulus) cells harvested from an adult mammal and dispersed mechanically and/or enzymatically (e.g., by hyaluronidase). The resulting dispersed cell suspension may be placed in a micromanipulation chamber facilitating detailed examination, selection and manipulation of individual cells to avoid those with certain characteristics (e.g., exhibiting advanced stages of apoptosis, necrosis or division). Gentle and repeated aspiration of cells selected in this way causes breakage of plasma membranes and allows the corresponding nucleus to be harvested. Individually selected nuclei are then aspirated into an injection pipette, described below, for insertion into enucleated oocytes.

In another embodiment of the invention, the donors of the adult cell nuclei are fibroblasts. Fibroblasts may be obtained from animals by methods well known to those skilled in the art. For example, fibroblasts may be obtained from adult mouse tails by placing minced tail tissue into short-term culture (e.g., 5-7 days at 37.5° C. under 5% CO₂ in air), during which time fibroblasts present in the culture become the predominant cell type. In further embodiments of the invention, thymus cells, spleen cells, macrophages are employed as the adult somatic cell nucleus donors. Methods for obtaining thymus or spleen cell suspensions are well known to those skilled in the art. Macrophages may be obtained, for example, by lavage of the peritoneal cavity or the lungs by methods known to those of skill in the art.

Other somatic cells that may be used as sources of nuclei include, without limitation, epithelial cells, neural cells, epidermal cells, keratinocytes, hematopoietic cells, melanocytes, chondrocytes, lymphocytes, monocytes, nucleated erythrocytes, Sertoli cells, cardiac muscle cells, skeletal muscle cells, smooth muscle cells, and other cells from organs including, without limitation, skin, lung, pancreas, liver, kidney, urinary bladder, stomach, intestine, and the like, (and, where appropriate, their progenitor cells), derived directly from in vivo sources, or following culture in vitro.

Insertion of Donor Nucleus into Enucleated Oocyte

Nuclei (or nuclear constituents including the chromosomes) may be injected directly into the cytoplasm of the enucleated oocyte by a microinjection technique. In a preferred method of injection of nuclei from somatic cells into enucleated oocytes, a piezo electrically-driven micropipette is used, in which one may essentially use the equipment and techniques described above (with respect to enucleation of oocytes), with modifications here detailed.

For example, an injection pipette is prepared, as previously described, such that it has a flat tip with an inner diameter of about 5 μm. The injection needle contains mercury near the tip and is housed in the piezo electrically-actuated unit according to the instructions of the vendor. The presence of a mercury droplet near the tip of the injection pipette increases the momentum and, therefore, penetrating capability. The tip of an injection pipette containing individually selected nuclei is brought into intimate contact with the zona pellucida of an enucleated oocyte and several piezo pulses (using controller setting scales of intensity 1-5, speed 4-6) are applied to advance the pipette while maintaining a light negative pressure within. When the tip of the pipette has passed through the zona pellucida, the resultant zona plug is expelled into the perivitelline space and the nucleus is pushed forward until it is near the tip of the pipette. The pipette tip is then apposed to the plasma membrane and advanced (toward the opposite face of the oocyte) until the holding pipette almost reaches the opposite side of the cortex of the oocyte. The oocyte plasma membrane is now deeply invaginated around the tip of the injection needle. Upon application of one to two piezo pulses (typically, intensity 1-2, speed 1), the oolemma is punctured at the pipette tip, as indicated by a rapid relaxation of the oolemma, which may be clearly visible. The nucleus is then expelled into the ooplasm with a minimum amount (about 6 pL) of accompanying medium. The pipette is then gently withdrawn, leaving the newly introduced nucleus within the cytoplasm of the oocyte. This method is performed briskly, typically in batches of 10-15 enucleated oocytes which at all other times are maintained in culture conditions.

Alternative microinjection variants, in which a conventional injection pipette is employed, may be used to insert the donor nucleus. An example of a suitable microinjection method employing a conventional pipette, for inserting sperm nuclei into hamster oocyte, is described in Yanagida, K., Yanagimachi, R., Perreault, S. D. and R. G. Kleinfeld, Biology of Reproduction 44, 440-447 (1991), the disclosure of which pertaining to such method is hereby incorporated by reference.

Activation of the Host Oocyte

In one embodiment of the invention, renucleated oocytes are returned to culture conditions for 0-6 hours prior to activation. Thus, in one embodiment of the invention, oocytes may be activated at any time up to approximately 6 hours (the latent period) after renucleation, either by electroactivation, injection of one or more oocyte-activating substances, or transfer of the oocytes into media containing one or more oocyte-activating substances.

Reagents capable of providing an activating stimulus (or combination of activating stimuli) include, but are not limited to, sperm cytoplasmic activating factor, and certain pharmacological compounds (e.g., Ca²⁺ and other signal transduction modulators), which may be introduced by microinjection after, or concomitantly with, renucleation. Some activating stimuli are provided following transfer of renucleated oocytes (either immediately or following a latent period) to media containing one or members of a sub-set of activating compounds, including stimulators of Ca²⁺ release (e.g., caffeine, Ca²⁺ ionophores such as A 23187 and ionomycin, and ethanol), modulators of phosphoprotein signaling (e.g., 2-aminopurine, staurospurine, and sphingosine), inhibitors of protein synthesis (e.g., A 23187, cyclohexamide), 6-dimethylaminopurine, or combinations of the foregoing (e.g., 6-dimethylaminopurine and ionomycin). In one embodiment of the invention, activation of mouse oocytes is achieved by culture for 1-6 hours in Ca²⁺-free CZB medium containing 2-10 mM Sr²⁺.

In embodiments of the invention wherein the activation stimulus is applied concurrently with or after renucleation, renucleated oocytes are transferred to a medium containing one or more inhibitors of microtubule and/or microfilament assembly (e.g., 5 μg/mL cytochalasin B) to inhibit extrusion of chromosomes (via a “polar body”) on or soon after application of the activating stimulus.

In one embodiment of the invention enucleated oocytes may be activated prior to renucleation. Activation methods may be as described above. Following exposure to an activating stimulus, oocytes may be cultured for up to approximately 6 hours prior to injection of a 2n somatic cell nucleus as described above. In this embodiment, somatically-derived chromosomes transform directly into pronucleus-like structures within the renucleated oocyte, and there is no need to suppress “polar body” extrusion by culture with a cytokinesis-preventing agent, such as cytochalasin-B.

Development of Embryos to Produce Viable Fetuses and Offspring

Following pronucleus formation, the embryo may be allowed to develop by culture in a medium that does not contain a microtubule or microfilament disrupting agent. Culture may continue to the 2-8 cell stage or morula/blastocyst stage, at which time the embryo may be transferred into the oviduct or uterus of a foster mother.

Alternatively, the embryo may be split and the cells clonally expanded, for the purpose of improving yield. Alternatively or additionally, it may be possible for increased yields of viable embryos to be achieved by means of the present invention by clonal expansion of donors and/or if use is made of the process of serial (nuclear) transfer, whereby nuclear constituents from resulting embryos may be transferred back into an enucleated oocyte, according to the method of the invention described above, to generate a new embryo. In a further embodiment of the invention, the pronuclear embryo is cultured in vivo following direct transfer into a suitable recipient.

Modulation of Cell Division or Embryonic Development

In one embodiment of the invention, renucleation of an oocyte permits the introduction, prior to, during, or after the combining of a nucleus with the enucleated oocyte, of one or more agents with the potential to alter the developmental outcome of the embryo. Alternatively or additionally, the agent(s) may be introduced prior to or following renucleation. For example, nuclei may be co-injected with antibodies directed against proteins with hypothetical regulatory roles with the potential to influence the outcome of the method of the invention. Such molecules may include, but are not limited to, proteins involved in vesicle transport (e.g., synaptotagmins), those which may mediate chromatin-ooplasm communication (e.g., DNA damage cell cycle check-point molecules such as chk1), those with a putative role in oocyte signaling (e.g., STAT3) or those which modify DNA (e.g., DNA methyltransferases). Members of these classes of molecules may also be the (indirect) targets of modulatory pharmacological agents introduced by microinjection and which have roles analogous to those of antibodies. Both antibodies and pharmacological agents work by binding to their respective target molecules. Where the target has an inhibitory effect on developmental outcome, this binding reduces target function, and where the target has a positive effect on developmental outcome, the binding promotes that function. Alternatively, modulation of functions important in the cloning process may be achieved directly by the injection of proteins (e.g., those in the classes above) rather than agents which bind to them.

In a further embodiment of the invention exogenous ribonucleic acid (RNA) or deoxyribonucleic acid (DNA) may be introduced into the oocyte by microinjection prior to or following renucleation. For example, injection of recombinant DNA harboring the necessary cis-active signals may result in the transcription of sequences present on the recombinant DNA by resident or co-injected transcription factors, and subsequent expression of encoded proteins with an antagonistic effect on development inhibitory factors, or with a positive effect on embryo development. Moreover, the transcript may possess antisense activity against mRNAs encoding development inhibitory proteins. Alternatively, antisense regulation may be achieved by injecting nucleic acids (or their derivatives) that are able to exert an inhibitory effect by interacting directly with their nucleic acid target(s) without prior transcription within the oocyte.

Recombinant DNA (linear or otherwise) introduced by the method of the invention may comprise a functional replicon containing one or more expressed, functional gene under the control of a promoter exhibiting anything from a narrow to a broad developmental expression profile. For example, the promoter might direct immediate, but brief expression where that promoter is active only in the early zygote. Introduced DNA may either be lost at some point during embryonic development, or integrate at one or more genomic loci, to be stably replicated throughout the life of the resulting transgenic individual. In one embodiment, DNA constructs encoding putative “anti-aging” proteins, such as telomerase or superoxide dismutase, may be introduced into the oocyte by microinjection. Alternatively, such proteins may be injected directly.

### EXAMPLES

The following examples illustrate the method of the invention and the development of live offspring from oocytes injected with adult somatic cell nuclei. In particular, the examples illustrate the cloning of mice from enucleated oocytes injected with nuclei isolated from adult mouse cumulus cells, Sertoli cells, neuronal cells, fibroblasts, spleen cells, thymus cells and macrophages. The examples described herein are intended to be only examples of animal oocytes, adult somatic cells, and media that may be used in the process of the invention, and are not intended to be limiting, as other examples of embodiments of the invention would readily be recognized by those skilled in the art.

Reagents

All inorganic and organic compounds were purchased from Sigma Chemical Co. (St. Louis, Mo.) unless otherwise stated.

The medium used for culturing oocytes after microsurgery was CZB medium (Chatot, et al., 1989. J. Reprod. Fert. 86, 679-688), supplemented with 5.56 mM D-glucose. CZB medium comprises 81.6 mM NaCl, 4.8 mM KCl, 1.7 mM CaCl₂, 1.2 mM MgSO₄, 1.8 mM KH₂PO₄, 25.1 mM NaHCO₃, 0.1 mM Na₂EDTA, 31 mM Na.lactate, 0.3 mM Na.pyruvate, 7 U/mL penicillin G, 5 U/mL streptomycin sulfate, and 4 mg/mL bovine serum albumin.

The medium for oocyte collection from oviducts, subsequent treatments and micromanipulation was a modified CZB containing 20 mM Hepes, a reduced amount of NaHCO₃ (5 mM) and bovine serum albumin at 3 mg/mL. This medium is herein termed Hepes-CZB. The pH of the CZB and Hepes-CZB media was approximately 7.5. For microinjection purposes, it was preferred to replace the BSA in the Hepes CZB with 0.1 mg/mL polyvinyl alcohol (PVA, cold water soluble, average molecular mass 10×10³) because PVA kept the wall of the injection pipette less sticky over a longer period of time than BSA and was beneficial during repeated use of a single pipette for multiple nuclei/oocyte transfers.

The medium used for activation of reconstituted oocytes was Ca²⁺-free CZB containing both 10 mM SrCl₂ and 5 μg/ml cytochalasin B. A stock solution of Sr²⁺ (100 mM in distilled water) was stored at room temperature. A stock solution of cytochalasin B (500 μg/ml in dimethylsulfoxide, DMSO) was stored at −20° C. Immediately before use, the Sr²⁺ stock solution was diluted 1:10 with Ca²⁺-free CZB such that the final concentration of Sr²⁺ was 10 mM. The cytochalasin B stock solution was diluted with Ca²⁺-free CZB such that the final cytochalasin concentration was 5 μg/ml in a final 1% DMSO concentration.

The medium used for isolation of brain cells was nucleus isolation medium (NIM), consisting of 123.0 mM KCl, 2.6 mM NaCl, 7.8 mM NaH₂PO₄, 1.4 mM KH₂PO₄, 3 mM Na₂EDTA. Its pH value was adjusted to 7.2 by addition of a small quantity of 1 M HCl. NIM supplemented with PVP (average molecular mass 3×10³, ICN Biochemicals, Costa Mesa, Calif.) was used to suspend the brain cells prior to injection.

Other media used in the examples are disclosed where appropriate.

Animals

B6D2F1 (C57BL/6×DBA/2), B6C3F1 (C57BL/6×C3H/He) and DBA/2 female mice, 5 to 10 weeks old, were used as oocyte donors. C57BL/6, C3H/He, DBA/2, B6D2F1 and B6C3F1 female mice, 5 to 10 weeks old, were used as the donors of cumulus cell nuclei. B6C3F1 male mice, 10 to 12 weeks old, were used as the donors of fibroblast cell nuclei. B6D2F1 male and female mice 5 to 10 weeks old, were used as the donors of other adult cell nuclei. Foster mothers were CD-1 females mated with vasectomized males of the same strain.

All animals used in these examples were maintained in accordance with the guidelines of the Laboratory Animal Service at the University of Hawaii and those prepared by the Committee on Care and Use of Laboratory Animals of the Institute of Laboratory Resources National Research Council (DHEW publication no. [NIH] 80-23, revised in 1985). The protocol of animal handling and treatment was reviewed and approved by the Animal Care and Use Committee at the University of Hawaii.

### Example 1

### Somatic Cell Preparation

In this example, cumulus cells from mouse oviducts were isolated for use as a source of adult somatic cell nuclei for injection into enucleated mouse oocytes. Derivations of the cloned mice produced in Series A-D of Table 2, and described below, are also described in Wakayama, et al., 1998, Nature 394, 369-374.

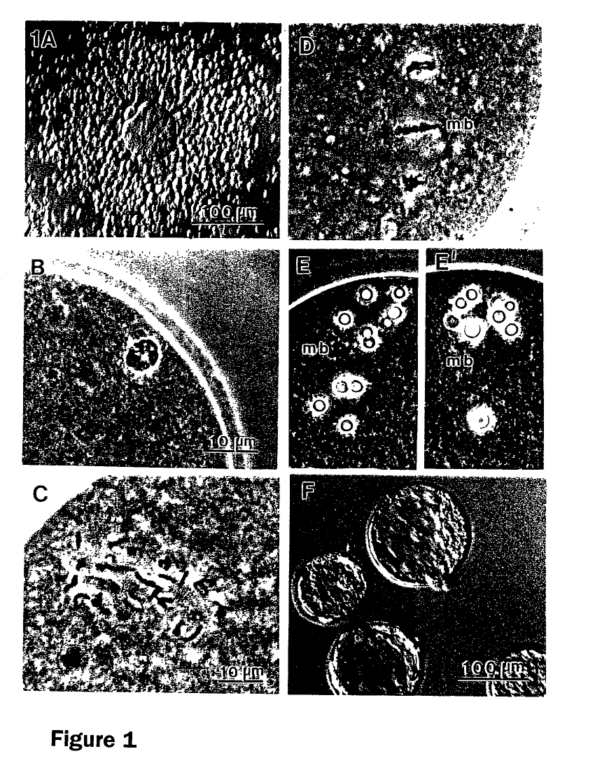

Female B6D2F1 (C57BL/6×DBA/2 used in Series A and B), B6C3F1 (C57BL/6×C3H/He used in Series C) or B6C3F1 cloned mice produced in Series D were induced to superovulate by consecutive intravenous injections of 5 to 7.5 units of equine chorionic gonadotrophin (eCG) and 5 to 7.5 units of human chorionic gonadotrophin (hCG). Thirteen hours after hCG injection, cumulus-oocyte complexes (see FIG. 1A) were collected from oviducts and treated in Hepes-CZB medium supplemented with bovine testicular hyaluronidase (0.1% [w/v], 300 U/mg, ICN Biochemicals, Costa Mesa, Calif.) to disperse cumulus cells. Medium sized cumulus cells (10-12 μm in diameter) were the most commonly found (>70%) and these were selected for injection. Following dispersal, cells were transferred to Hepes-CZB containing 10% (w/v) polyvinylpyrrolidone (average molecular weight, 360,000 daltons) and retained at room temperature for up to 3 hours prior to injection.

### Example 2

### Somatic Cell Preparation

In this Example, Sertoli cells and brain cells (neurons) were isolated from adult mice. These cells characteristically do not divide in adult animals and remain permanently in G0 phase of the cell cycle.

Seminiferous tubules were isolated from the testis and exposed for 20 minutes at 30° C. to a solution of 1 mg collagenase per ml of Hepes-CZB. Tubules were then minced with a razor blade and placed in Hepes-CZB containing trypsin at 1 mg/ml with occasional agitation. The resultant suspension was then allowed to stand. The Sertoli cell rich fraction settled first. Suspended cells were removed by aspiration and fresh medium used to resuspend the remainder. Sertoli cells, with characteristic morphological features, are readily identifiable under low power microscopy. Manipulation of individual Sertoli cells was performed using a large injection pipette (inner diameter≈10 μm).

Neuronal cells were isolated from the cerebral cortex of adult B6D2F1 females. Brain tissue was removed with sterile scissors, quickly washed in erythrocyte-lysing buffer and gently hand-homogenized for a few seconds in nucleus isolation medium (NIM) at room temperature. Nuclei harboring a conspicuous nucleolus were individually collected from the resulting suspension using the injection pipette, prior to delivery into a recipient enucleated oocyte.

### Example 3

### Somatic Cell Preparation

Fibroblast cells were prepared from tails of adult B6C3F1 mice. The tail was isolated from a mouse, freed from its skin, cut into small pieces, and placed into a tissue culture dish in 5 ml Dulbecco's Modified Eagle's Medium (DMEM, Sigma) supplemented with 10% fetal calf serum (FCS, Hyclone, Logan, Utah). After 5 to 7 days of incubation at 37.5° C. under 5% CO₂ in air, many fibroblasts were seen spreading along the inner surface of the dish. In some experiments, the medium in the dish was replaced with FCS-free DMEM and cultured for an additional 3 to 5 days. To detach fibroblasts from the dish, the medium was replaced with Ca²⁺-free, Mg²⁺-free phosphate buffered saline (PBS) containing 0.25% trypsin and 0.75 mM ethylenediaminetetraacetic acid (EDTA, Specialty Media, Lavallette, N.J.). Ten minutes later, the medium was agitated by pipetting for a few minutes to release the cells from the surface of the dish. The medium was collected and centrifuged (150×g for 10 minutes) to sediment the cells. The cells were then washed three times by centrifugation in DMEM medium.

### Example 4

### Somatic Cell Preparation

Spleens were removed from adult male and female B6D2F1 mice. After blood adhering to the surface was removed by washing in CZB medium, each spleen was placed in 5 ml of Hepes-CZB medium and tom into small pieces to allow the cells to disperse into the medium.

### Example 5

### Somatic Cell Preparation

Thymuses were removed from adult male and female B6D2F1 mice. After blood adhering to the surface was removed by washing in CZB medium, each thymus was placed in 5 ml. of Hepes-CZB medium and torn into small pieces to allow the cells to disperse into the medium.

### Example 6

### Somatic Cell Preparation

Immediately after a female or male (B6D2F1) mouse was euthanized, 5 ml of 0.9% NaCl or CZB medium was injected, through a hypodermic needle, into its peritoneal cavity. The abdomen was then massaged and the medium recovered through the needle. The recovered medium containing peritoneal macrophages was centrifuged to sediment the cells. The cells were then resuspended in Hepes-CZB medium.

### Example 7

### Enucleation of Mature Unfertilized Oocytes

In this Example, murine Met II oocytes were harvested, enucleated, and subsequently microinjected with nuclei isolated from the cells of Examples 1 through 6, using a piezo electrically-actuated micropipette. All oocyte manipulations, culture, and insertions of cell nuclei were performed under a layer of mineral oil, preferably containing Vitamin E as an antioxidant, such as that available from E.R. Squibb and Sons, Princeton, N.J.

Enucleation of the oocytes was achieved by aspiration with a piezo electric-driven micropipette using the Piezo Micromanipulator Model MB-U by Prime Tech Ltd. (Tsukuba, Ibaraki-ken, Japan). This unit uses the piezo electric effect to advance the pipette holder a very short distance (approximately 0.5 μm) at a time at a very high speed. The intensity and speed of the pulse were regulated by the controller.

Oocytes (obtained 13 hours post hCG injection of eCG-primed females) were freed from the cumulus oophorus and held in CZB medium at 37.5° C. under approximately 5% (v/v) CO₂ in air until required. A group of oocytes (usually 15-20 in number) was transferred into a droplet (about 10 μl) of Hepes-CZB containing 5 μg/ml cytochalasin B, which had been previously placed in the operation chamber on the microscope stage. After an oocyte was held by an oocyte-holding pipette, its zona pellucida was “drilled” by applying several Piezo-pulses to the tip of an enucleation pipette (about 10 μm in inner diameter). The Met II chromosome-spindle complex, distinguished as a translucent spot in the ooplasm, was drawn into the pipette with a small amount of accompanying ooplasm, then gently pulled away from the oocyte until a stretched cytoplasmic bridge was pinched off. After all oocytes in one group (usually 20 oocytes) were enucleated (which took about 10 minutes), they were transferred into cytochalasin B-free CZB and kept there for up to 2 hours at 37.5° C. As assessed by fixing and staining the oocytes, as described above, the efficiency of enucleation was 100%.

### Example 8

### Insertion of Adult Somatic Cell Nuclei into Enucleated Oocytes

For injection of donor nuclei into the enucleated oocytes prepared as described above, a microinjection chamber was prepared by employing the cover (approximately 5 mm in depth) of a plastic dish (100 mm×15 mm; Falcon Plastics, Oxnard, Calif., catalogue no. 1001). A row consisting of two round droplets and one elongated drop was placed along the center line of the dish. The first droplet (approximately 2 μL; 2 mm in diameter) was for pipette washing (Hepes-CZB containing 12% [w/v] PVP, average molecular weight, 360,000 daltons). The second droplet (approximately 2 μL; 2 mm in diameter) contained a suspension of donor cells in Hepes-CZB. The third elongated droplet (6 μL; 2 mm wide and 6 mm long) was of Hepes-CZB medium for the oocytes. Each of these droplets were covered with mineral oil. The dish was placed on the stage of an inverted microscope with Hoffman Modulation contrast optics.

Microinjection of donor cell nuclei into oocytes was achieved by the piezo electric microinjection method described previously. Nuclei were removed from their respective somatic cells and subjected to gentle aspiration in and out of the injection pipette (approximately 7 μm inner diameter) until their nuclei became “naked” or almost naked (i.e., largely devoid of visible cytoplasmic material). For cells with “tough” plasma membranes (e.g., tail fibroblasts), a few Piezo pulses were applied to break the membranes. After the “naked” nucleus was drawn deeply into the pipette, the next cell was drawn into the same pipette. These nuclei were injected one by one into enucleated oocytes.

For nucleus injection, a small volume (about 0.5 μL) of mercury was placed near the proximal end of the injection pipette, which was then connected to the piezo electric-driven unit described above. After the mercury had been pushed towards the tip of the pipette, a small volume of medium (approximately 10 pL) was sucked into the pipette.

An enucleated oocyte was positioned on a microscope stage in a drop of CZB medium containing 5 μg/mL cytochalasin B. The oocyte was held by a holding pipette while the tip of the injection pipette was brought into intimate contact with the zona pellucida. Several piezo pulses (e.g., intensity 1-2, speed 1-2) were given to advance the pipette while a light negative pressure was applied within it. When the tip of the pipette had passed through the zona pellucida, the cylindrical piece of the zona in the pipette was expelled into the perivitelline space. After the donor nucleus was pushed forward until it was near the tip of the injection pipette, the pipette was advanced mechanically until its tip almost reached the opposite side of the oocyte's cortex. The oolemma was punctured by applying 1 or 2 piezo pulses (typically, intensity 1-2, speed 1) and the nucleus was expelled into the ooplasm with a minimum volume (about 6 pL) of accompanying medium. Sometimes, as possible of the medium was retrieved. The pipette was then gently withdrawn, leaving the nucleus the ooplasm. Each oocyte was injected with one nucleus. Approximately 5-20 oocytes were microinjected by this method within 10-15 minutes. All injections were performed at room temperature usually in the range of 24°-28° C. All manipulations were performed at room temperature (24° to 26° C.). Each nucleus was injected into a separate enucleated oocyte within less than 10 minutes after denudation.

FIG. 1B illustrates a cumulus cell nucleus in an enucleated oocyte within 10 minutes of injection.

The nuclei of Sertoli cells and brain cells, prepared as described in Example 2, were also injected by piezo electric microinjection into enucleated oocytes, by the method described above for the injection of cumulus cells.

The nuclei of tail fibroblasts, spleen cells, thymus cells and macrophages, prepared as described in Examples 3, 4, 5, and 6, respectively, were also injected by piezo electric microinjection into enucleated oocytes, by the method described above for the injection of cumulus cells.

Some oocytes containing an injected nucleus were then immediately activated as described in Example 9. Other similar oocytes were incubated for a time period of up to about 6 hours prior to activation.

### Example 9

### Oocyte Activation

Following somatic cell nucleus injection, some groups of oocytes were placed immediately in Ca²⁺-free CZB containing both 10 mM Sr²⁺ and 5 μg/mL cytochalasin B for 6 hours. Additional groups of enucleated oocytes injected with cumulus cell nuclei were left in CZB at 37.5° C. under 5% (v/v) CO₂ in air for about 1 to about 6 hours, preferably about 1 to about 3 hours, during which time the nucleus within the oocyte decondensed and transformed into visible chromosomes {is this stated correctly?}. The oocytes were then incubated for about 6 to about 7 hours in Ca²⁺-free CZB containing both 10 mM Sr²⁺ and 5 μg/mL cytochalasin B for 6 hours for activation. Sr²⁺ treatment activated the oocytes, while the cytochalasin B prevented subsequent polar body formation and, therefore, chromosome expulsion, thus assuring that all the chromosomes of the adult somatic cell nucleus remained in the cytoplasm of the activated oocyte. Examination of enucleated oocytes injected with cumulus cell nuclei revealed that chromosome condensation had occurred within 1 hour following injection (see FIG. 1C). When, subsequent to 1 to 6 hours incubation in Sr²⁺-free medium, oocytes were activated in culture medium containing Sr²⁺ and cytochalasin B, their cumulus-derived chromosomes segregated (see FIG. 1D) to form structures resembling the pronuclei formed after normal fertilization (referred to here as pseudo-pronuclei). Examination of 47 such oocytes after fixation and staining showed that 64% had two pseudo-pronuclei (see FIGS. 1E and 1E′) and 36% had three or more. Oocytes with at least one distinct pseudo-pronucleus were considered normally activated. Chromosome analysis of 13 such oocytes fixed prior to the first cleavage (data not shown) revealed that 85% had a normal diploid chromosome number (2n=40).

Activated oocytes were washed and cultured in Sr²⁺- and cytochalasin B-free CZB medium until they reached the 2- to 8-cell or morula/blastocyst stage at 37.5° C. under 5% (v/v) CO₂ in air.

FIG. 1F illustrates live blastocysts produced following injection of enucleated oocytes with cumulus cell nuclei.

### Example 10

### Embryo Transfer

Two- to eight-cell embryos (24 hours or 48 hours after the onset of activation) were transferred into oviducts or uteri of foster mothers (CD-1, albino) that had been respectively mated with vasectomized CD-1 males 1 day previously. Morulae/blastocysts (72 hours after activation) were transferred into uteri of foster mothers mated with vasectomized males 3 days previously. When cumulus cells or fibroblasts were used as nucleus donors, recipient females were euthanized at 19.5 dpc and their uteri were examined for the presence of fetuses and implantation sites. Live fetuses, if any, were raised by other lactating foster mothers (CD-1). When other somatic cell nuclei (i.e., spleen and thymus cells and macrophages) were used, all recipient females were euthanized at 8.5 to 12.5 dpc, and their uteri were examined for the presence of fetuses and implantation sites.

### Example 11

### DNA Typing

DNA from the following control strains and hybrids was obtained from spleen tissue: C57BL/6J (B6), C3H/HeJ (C3), DBA/2J (D2), B6C3F1 and B6D2F1. DNA from the three cumulus cell donor females (B6C3F1), the three oocyte recipient females (B6D2F1), and the three foster females (CD-1) was prepared from tail tip biopsies. Total DNA from six B6C3F1-derived, cloned offspring was prepared from their associated placentas.

For the microsatellite markers D1Mit46, DS2Mit102, and D3Mit49, primer pairs (MapPairs) were purchased from Research Genetics (Huntsville, Ala.) and typing performed as previously described in Dietrich, W. et al., Genetics 131, 423-447 (1992), except that PCR reactions were carried out for 30 cycles and products were separated by 3% agarose gels (Metaphor) and visualized by ethidium bromide staining.

The identification of endogenous ecotropic murine leukemia provirus DNA sequences (Emv loci) was following hybridization of PvuII-digested genomic DNA to the diagnostic probe, pEc-B4, according to the method described in Taylor, B. A. and L. Rowe, Genomics 5, 221-232 (1989). Probe labeling, Southern blotting, and hybridization procedures were as previously described in Johnson, K. R. et al., Genomics 12, 503-509 (1992).

### Example 12

### Examination of Placenta

When full term fetuses (19.5 dpc) were found in uteri, placentas were isolated, weighed and fixed with Bouin's solution for later examination of histological details. In general, only one or two of the implanted cloned mouse offspring reached term in each of the host foster mothers. During the course of the present study, it was noticed that the placenta of cloned fetuses are significantly larger than those of normal fetuses (see Table 7). To investigate the possibility that the large placenta may be due to the small number of fetuses in each uteri (during a normal pregnancy, each mouse uterus carries several, or as many as ten, fetuses), the litter size of normal pregnancies was purposely reduced, as follows: C57BL/6 female mice were mated with C3H/He males. The next day, eggs containing pronuclei were collected from the oviduct, and 2 to 3 eggs were transferred to the oviducts of each pseudo-pregnant foster mother (CD-1) in order to allow the implantation of only 1 to 2 embryos. The embryos and placentas were weighed on 19.5 dpc.

### Results

Cloning with Cumulus Cell Nuclei.

The preimplantation development of host enucleated oocytes injected with the nuclei from cumulus cells is illustrated in Table 1. Out of 182 oocytes subjected to an activating stimulus immediately after injection, 153 (84.1%) were successfully activated and survived. Of these 153 oocytes, 61 developed into morula/blastocysts in vitro. However, 474 (93.3%) out of 508 injected oocytes activated 1-3 hours after injection, and 151 (83.0%) out of 182 injected oocytes activated 3-6 hours after injection, were successfully activated and survived. Of these, 277 (58.4%) and 101 (66.9%), respectively, developed into morula/blastocysts in vitro. Therefore, significantly higher proportions of oocytes developed into morula/blastocysts in vitro when they were activated 1-6 hours after nucleus injection, as compared to oocytes activated immediately after injection (p<0.005), and the time interval between nucleus injection and oocyte activation in these experiments appears to affect the rate of oocyte development.

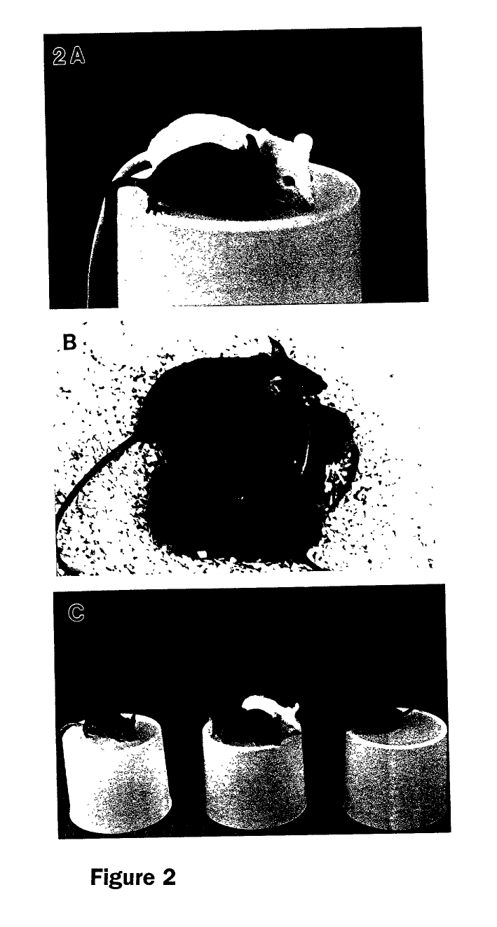

The development of host enucleated oocytes injected with the nuclei of cumulus cells is illustrated in Table 2. In the first series of experiments (Series A), a total of 142 developing embryos (at 2-cell to morula/blastocyst stage) were transferred to 16 recipient females. When these females were examined on day 8.5 and 11.5 day post coitum (dpc), 5 live and 5 dead fetuses were seen in uteri. In the second series of experiments (Series B), a total of 800 embryos were transferred into 54 foster mothers. When Cesarean sections were performed on 18.5-19.5 dpc, 17 live fetuses were found. Of these, 6 died soon after delivery, 1 died approximately 7 days after delivery, but the remaining 10 females survived and are apparently healthy. All of these, including the first-born (named “Cumulina”, in the foreground of the photograph, FIG. 2A, with her albino foster mother) have been mated and delivered and raised normal offspring. FIG. 2B is a photograph of Cumulina at 2.5 months with the pups she produced following mating with a CD-1 (albino) male. Several of these offspring have, in turn, now developed into fertile adults.

In a third series of experiments (Series C in Table 2), B6C3F1 cumulus cell nuclei were injected into enucleated B6D2F1 oocytes. Whereas B6D2F1 mice are black, B6C3F1 mice carry a copy of the agouti A gene, and are consequently agouti. Offspring from this experiment should therefore have an agouti coat color, rather than the black of the B6D2F1 oocyte donors. A total of 298 embryos derived from B6C3F1 cumulus cell nuclei were transferred to 18 foster mothers. Cesarean sections performed 19.5 dpc revealed 6 live fetuses whose placentas were used in DNA typing analysis (see Example 6 above). Although 1 died a day after birth, the 5 extant females are healthy and have the agouti coat phenotype. FIG. 2C shows two such cloned agouti pups with their albino foster mother (CD-1) in the center of the photograph. To the left of the photograph is the corresponding agouti B6C3F1 cumulus donor. The cloned pups (center) are like the identical ‘twin’ sisters (i.e., they are the clones) of the cumulus donor. The B6D2F1 oocyte donor (black) is shown in the right of the photograph.

Additional experiments (Series D in Table 2) were performed to investigate whether clones could be efficiently cloned in subsequent rounds of recloning. In this experiment, cumulus cells were harvested from B6C3F1 (agouti) clones generated in Series C, and their nuclei were injected into enucleated B6D2F1 oocytes to generate embryos that were transferred as described for Series A-C. A total of 287 embryos derived from cloned B6C2F1 cumulus cell nuclei were transferred to 18 foster mothers. When Cesarean sections were performed 19.5 dpc, 8 live fetuses were recovered. Although 1 died soon after birth, the 7 surviving females are healthy and have the expected agouti coat phenotype. These results suggest that clones (Series B and C) and cloned clones (Series D) are produced with a similar efficiency. Subsequently, it has been possible to repeat the process using animals from Series D (data not shown) as cumulus chromosome donors, resulting in the birth of cloned clones (third generation clones). Therefore, it appears that successive generations of clones do not undergo changes (either positive or negative) that influence the outcome of the cloning process.

Confirmation of Genetic Identity of Clones to Cumulus Cell Donors.

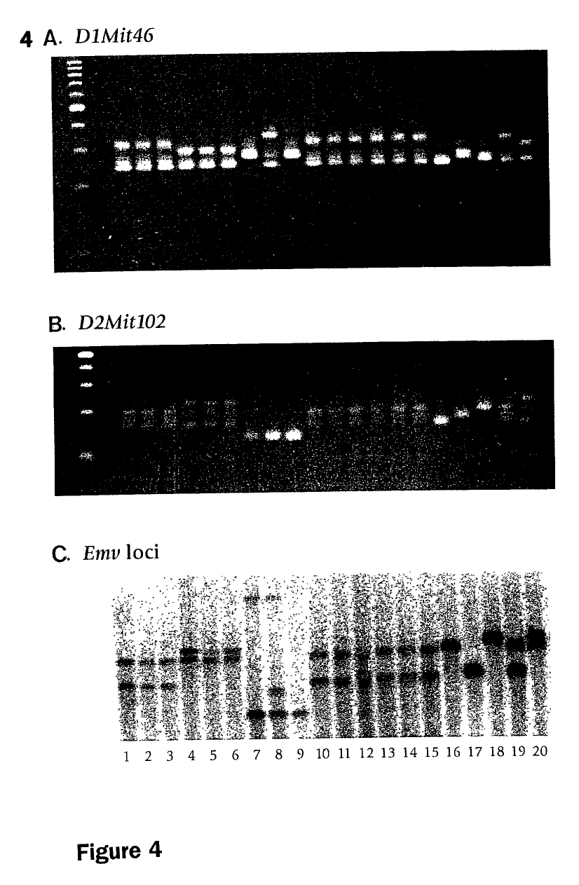

As illustrated in FIGS. 4A, 4B and 4C, DNA typing of donors and offspring in Series C corroborates the genetic identity of cloned offspring to cumulus cell donors, and non-identity to oocyte donors and host foster females. PCR typing of DNA was employed, using highly variable alleles (strain-specific markers) diagnostic of the C57BL/6, C3H, DBA/2 and CD-1 mouse strains. These strains, or their F1 hybrids, were used in this work and they therefore collectively account for all of the genotypes present. In all of the Figures, placental DNA from the six cloned Series C offspring (lanes 10-15) was compared with DNA from the three cumulus cell donor females (B6C3F1, lanes 1-3), the three oocyte donor females (B6D2F1, lanes 4-6), and the three host females (CD-1, lanes 7-9). Control DNA was from C57BL/6 (lane 16), C3H (lane 17), DBA/2 (lane 18), B6C3F1 (lane 19) and B6D2F1 (lane 20). FIGS. 4A and 4B illustrate the results of DNA typing employing agarose gels and the strain-specific markers D1Mit46 and D2Mit102, and FIG. 4C illustrates the results of DNA typing employing Southern blot analysis and the strain-specific Emv loci (Emv1, Emv2 and Emv3) markers.

The data presented in these Figures show genetic superimposability between cumulus nucleus donors and putative clones, and genetic non-identity with either the oocyte donors or the foster mothers. Therefore, the genome of each of the six cloned mice was derived from the nucleus of a cumulus cell.

That all of the live offspring reported here in Series B-D represent clones derived exclusively from the chromosomes of cumulus cells is confirmed in several ways. (1) The oocytes/eggs were not exposed to spermatozoa in vitro. (2) Foster mothers (CD-1, albino) were mated with vasectomized males (CD-1, albino) of proven infertility. In the unlikely event of fertilization by such a vasectomized male, the offspring would be albino. (3) The 2-8 cell embryos or blastocysts were transferred into oviduct/uteri of foster mothers. It is well established that 2-8 cell mouse embryos/blastocysts are totally refractory to fertilization by spermatozoa. (4) All term animals were born with black eyes. The surviving 10 from Series B have black coats and the surviving 5 in Series C have agouti coats. This pattern of coat color inheritance exactly matches that predicted by the genotype of the nucleus donor in each case. Since B6D2F1 mice lack the agouti A gene, the agouti mice in Series C must have inherited their agouti coat color from a non-B6D2F1 nucleus. (5) DNA typing of highly variable alleles diagnostic of the B6, C3, D2 and CD-1 strains used here (FIG. 4) demonstrates beyond reasonable doubt that the six cloned offspring in Series C (which includes one that died soon after birth) are isogeneic with the three cumulus cell donor females used (B6C3F1) and do not contain DNA derived from either the oocyte donors (B6D2F1) or host foster mothers (CD-1). (6) Following enucleation, extrusion of chromosomes into polar bodies was suppressed by using cytochalasin B. Thus, if enucleation of the oocytes had been totally unsuccessful or only partially successful, all embryos would have been hyperploid and would not have developed into normal offspring. (7) In mock experiments, in which 204 oocytes were enucleated and examined after fixation and staining, no chromosomes were apparent, suggesting the efficiency of chromosome removal exceeded 99.99%.

In Example 1, the cell type used was identified as the cumulus cell, with a high degree of certainty. The cells were not cultured in vitro. Ample time was given for cumulus nuclei to transform into condensed chromosomes within the cytoplasm of enucleated Met II oocytes. The rate of embryo development to morulae/blastocysts and implantation was very high. Prolonging the time between nuclear injection and oocyte activation was beneficial for both pre-implantation and post-implantation development (see Tables 1 and 2) and may have enhanced the opportunity of cumulus cell genes to undergo reprogramming for embryonic development.

It is believed that the use of a piezo electric micromanipulator also contributed to a higher rate in embryonic development. This apparatus allowed manipulation of oocytes and donor cells (e.g., drilling the zona pellucida to enucleate the oocyte, and injecting of donor cell nuclei) to be performed very quickly and efficiently. Introduction of donor nuclei into oocytes using a piezo electric driven pipette appears to be less traumatic to the oocytes than the use of an electric pulse, Sendai virus or polyethylene glycol, and allows for introduction of the somatic cell nucleus directly into the cytoplasm of the oocyte. Also, the amount of somatic cell cytoplasm introduced into enucleated oocytes was minimized by microinjection. This may also have contributed to the high preimplantation development of embryos in the present invention.

Cloning with Sertoli and Brain Cell Nuclei.

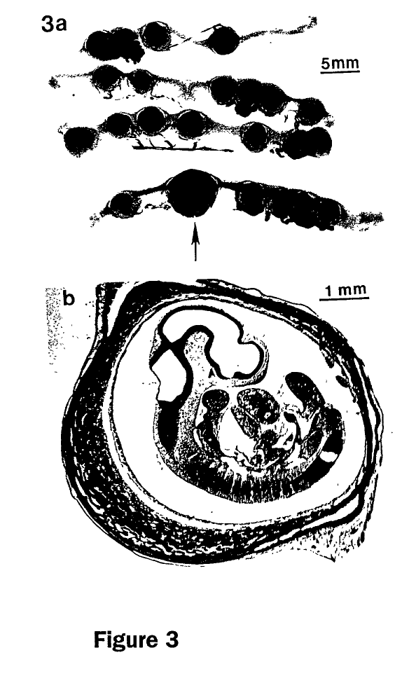

About 63 (40%) and 50 (22%) of enucleated oocytes injected with Sertoli cell nuclei and brain cell nuclei, respectively, developed into morulae/blastocysts in vitro and, of these 59 and 46, respectively were transferred to uteri of recipient foster mothers. FIG. 3 illustrates development of transferred embryos following injection of Sertoli cell nuclei into enucleated oocytes. FIG. 3A is a photograph of the uteri of recipient at 8.5 dpc. However, all uterine implantation sites failed to develop except for one live fetus (FIG. 3B) was found in the uterus of a foster mother euthanized 8.5 dpc (Table 3). None of the enucleated oocytes injected with brain cell nuclei developed beyond 6-7 dpc (Table 3). Thus, the method of the invention provided embryonic and fetal development of oocytes injected with the nuclei of Sertoli cells or brain cells.

Cloning with Adult Fibroblast Nuclei.

The results of experiments in which the nuclei of fibroblasts from the tails of B6C3F1 adult males (agouti) were injected into enucleated oocytes of B6D2F1 females (non-agouti) are illustrated in Table 5. As illustrated, about 50% of the activated oocytes injected with fibroblasts cultured in serum-containing medium developed to the morula/blastocyst stage. Of these, 177 2-cell or morula/blastocyst stage embryos were transferred to recipient foster mothers, and 1.1% of the embryos reached full term (i.e., 2 live offspring were born). About 58% of the activated oocytes injected with fibroblasts cultured in serum-free medium developed to the morula/blastocyst stage. Of these, 97 2-cell or morula/blastocyst stage embryos were transferred to recipient foster mothers, and 1.0% of the embryos reached full term (i.e., 1 live offspring was born). All live offspring were males and had black eyes and agouti coat color, as did the donors of the fibroblast nuclei. All of the above offspring proved to be fertile when mated. Whether or not the fibroblasts were cultured in serum-free medium or medium with serum appeared to make little or no difference in the number of live offspring obtained.

Cloning with Adult Spleen, Thymus and Macrophage Nuclei.

The development of enucleated oocytes receiving nuclei of adult spleen, thymus or macrophage cells is also illustrated in Table 4. In these studies, thymus cells supported the development of 3.1% of activated oocytes to morulae/blastocysts, but none developed beyond this stage.

Spleen cell nuclei supported embryonic development of 21% to 22% of activated oocytes to the morula/blastocyst stage. Although many implanted after transfer, they appeared to be resorbed by 6 to 7 dpc.

Macrophage nuclei supported embryonic development of 23% to 31% of activated oocytes to morulae/blastocysts, but embryos were absorbed or stopped their development before 6 to 7 dpc.

Thus, the method of the invention provided embryonic and fetal development of oocytes injected with the nuclei of thymus, spleen or macrophage cells. Since, in these studies, thymus, spleen and macrophage nuclei from adult animals showed more limited support for embryonic development than cumulus cell nuclei or fibroblast nuclei, it appears likely that nuclei from these cells may support the development of live offspring, but at a lower efficiency than nuclei from other adult cells.

Cloning with Cumulus Cell Nuclei from Inbred and Hybrid Strains of Mice.

Experiments were performed in which cumulus cell nuclei from three different inbred strains and two hybrid strains of the mouse were injected into enucleated oocytes. The results are illustrated in Table 6. When cumulus cells of inbred mice (C57BL/6, C3H/He and DBA/2) were injected into hybrid (B6D2F1) oocytes, some oocytes developed into normal-looking blastocysts, and one (DBA/2×B6D2F1)developed to a full-term live offspring. In contrast, a total of 41 live offspring (2%-4% of transferred embryos) were obtained when cumulus cell nuclei from hybrid B6D2F1 and B6C3F1 mice were injected into enucleated oocytes of the same hybrid mice, respectively. These offspring were all females. They had black eyes and the same coat color as the donors of the cumulus cell nuclei.

Differences in the Placental Weight of Cloned vs. Normal Mouse Pregnancies.

During the course of our study, a marked difference between pregnancies with cloned mice and normal mice was noticed, with respect to the weight of the placenta. As illustrated in Table 7, the mean weight of the placenta of cloned mice was 0.25 to 0.33 grams, whereas that of the control (normal) placenta having the same number of fetuses was about 0.12 to 0.15 grams, which was about half of the weight of the cloned mice placenta.

We believe that all the live offspring reported here represent clones derived from adult somatic cell nuclei, particularly cumulus cells and fibroblasts, in the absence of genetic contamination for the following reasons: (1) Oocytes/eggs were never exposed to spermatozoa in vitro during the course of the experiments. In mammals, intact oocytes cannot develop to term without spermatozoa. (2) Foster mothers (CD-1) were mated with vasectomized males (CD-1, albino) of proven infertility. Even if vasectomized males ejaculated spermatozoa and fertilized CD-1 oocytes, all of their offspring should be albino. Reconstructed 2- to 8-cell embryos or blastocysts were transferred into oviducts/uteri of foster mothers. Such developing embryos will never be fertilized by spermatozoa even if vasectomized males ejaculated spermatozoa. (3) All full-term animals were born with black eyes (not albino) and the pattern of coat color inheritance exactly matches that predicted by the genotype of the nucleus donor in each case. B6D2F1 mice lack the agouti gene which was used for oocyte recipients. Therefore the only way to obtain agouti offspring is via the donor cell nucleus (e.g., tail fibroblasts and some cumulus cells) from the B6C3F1 mice. (4) The sex of the cloned mice was consistent with the sex of the donor mice. Clones derived from female cumulus cells were all female. Clones derived from male tail fibroblasts were all male. (5) The extrusion of chromosomes into polar bodies was suppressed by the use of cytochalasin B. Thus, even if enucleation of the oocytes had been totally unsuccessful or only partially successful, all zygotes would have been hyperploid; such embryos cannot develop into normal offspring.

It has been demonstrated herein that the method of the invention can be used to obtain live, cloned mouse offspring from adult cumulus cell and adult fibroblast cell nuclei. The success rate has been up to 3%. To date, the method has been the most successful with the nuclei of cumulus cells. The reasons for this are not clear. Each mouse oocyte is surrounded by about five thousand cumulus cells (data not shown). It is known that the cumulus cells all communicate with each other via gap junctions throughout follicular development. Those closest to the oocyte (corona radiata cells) are in contact with the oocyte via gap junctions. Without being bound by theory, it is thought to be conceivable that significant exchanges of ions and small molecules (<2,000 Mr) occur between the oocyte and surrounding cumulus cells. This may affect cumulus cell genes, such that the genome becomes more readily “reprogramrnable” within the cytoplasm of an enucleated oocyte.

It was found that the best cloning results were obtained by the method of the invention when cumulus cell nuclei of hybrid mice were injected into enucleated oocytes of the corresponding hybrid mice. The only exception was the case in which dBA/2 cumulus cell nuclei were injected into hybrid (B6D2F1) oocytes. Why cumulus cell nuclei of inbred mice commonly failed to support postimplantation development of embryos is not known at this time. Mann and Stewart (Development 113, 1325-1333 (1991)) reported that the developmental potential of androgenetic aggregation chimeras is to some extent dependent on the mouse strain. Moreover, it is well known that the embryos of mouse hybrids are much easier to culture in vitro than those of inbred mice. (Suzuki, et al. (1996) Reprod. Fertil. Dev. 8, 975-980). It appears that heterosis facilitates the development of cloned embryos to term.

Three live cloned mice were produced by the method of the present invention using fibroblasts of adult males. It has previously been claimed that the key success to clone sheep was to bring a donor cell to G0 phase of the cell cycle. For example, Wilmut et al. did this by culturing cells in serum-free medium to “starve” them. In the present experiments, there did not appear to be a marked beneficial effect of culturing adult fibroblasts in serum-free medium to increase the success rate of cloning. It has also been reported that cloned calves were obtained from fetus cells cultured with serum (Cibelli et al., Science 280, 1256-1258 (1998)). It appears that an actively dividing population of cells can support development to term after nuclear transfer and that serum starvation is not a necessary treatment, at least in the mouse model.

In these experiments, it was noted that all cloned fetuses had large placentas, almost twice as large as normal placentas. Occasionally, a large placenta without a discernable fetus was found (data not shown). Large placentas were also noted by Kono et al. (Nature Genet. 13, 91-94 (1996)) in diploid parthenogenetic embryos developed from a mature oocyte fused with a very young, small oocyte. At day 13.5 of gestation, these parthenogenetic embryos had excessively large placentas. Kono et al. suggested that the lack of expression of genes from maternal alleles may explain the increased embryonic and placental development compared to normal mice. Some other genes may be important specifically for placental development, such as the maternally expressed Mash 2 (Guillemot et al., (1995). Nature Genet. 9, 235-242), and paternally expressed (maternally repressed) genes necessary for proliferation of the polar trophectoderm cells (Barton, et al., (1985). J. Embryol. Exp. Morphol. 90, 267-285). In addition, some cloned mice that died just after birth had a larger weight than others. Further, as reported by Kato, et al., (Science 282, 2095-2098 (1998)), dead cloned calves derived from somatic cells tended to be larger than the live ones. This would suggest that during nuclear reprogramming of somatic cells after nuclear transfer, some genes were not completely finished or reprogrammed to work normally. Without being bound by theory, these findings would be consistent with possible changes in imprinted gene expression.

While the invention has been described herein with reference to the preferred embodiments, it is to be understood that it is not intended to limit the invention to the specific forms disclosed. On the contrary, it is intended to cover all of the manifold modifications and alternative forms falling within the spirit and scope of the invention.

TABLE 1
Preimplantation Development of Enucleated Mouse Eggs Injected With Cumulus Cell Nuclei

| | | | | | No.(mean % ± | | |
|---|---|---|---|---|---|---|---|
| | Total No. | | No. of | | SD) of embryo developed from | | |
| | of | No. of | surviving | | activated oocytes, at 72 h after activation | | |
| Time of oocyte | oocytes | enucleated | oocytes after | No.(%) of | 1-cell and | | |
| activation | used | oocytes | injection | activated oocytes | abnormal | 2 to 8-cell | Morula/Blastocyst |
| Simultaneously | 233 | 230 | 182 | 153(84.1) | 17 | 75 | 61(39.9 ± 116.6)^{a} |
| with injection | | | | | | | |
| 1-3 hour after | 573 | 565 | 508 | 474(93.3) | 20 | 177 | 277(58.4 ± 12.6)^{b} |
| injection | | | | | | | |
| 3-6 hour after | 195 | 191 | 182 | 151(83.0) | 9 | 41 | 101(66.9 ± 14.4)^{b} |
| injection | | | | | | | |

Superscripts ^{a} or ^{b} within the same column indicate significant difference between(P < 0.005). The data were analyzed by the Chi-square test.

TABLE 2
Postimplantation Development of Enucleated Mouse Eggs Injected With Cumulus Cell Nuclei

| | | | No. | No.(%) | No. fetuses developed from transferred | | | | | No.(%) |
|---|---|---|---|---|---|---|---|---|---|---|
| | | No. | transferred | implantation | embryos | | | | | newborn from |
| Exp. | Time of oocyte | injected | embryos | from transferred | Total | 8.5 dpc | | 11.5 dpc | | transferred |
| series.* | activation | oocyte | (Recipients) | embryos^{†} | (%)^{†} | Live | Dead | Live | Dead | embryos |
| A | Simultaneously | 82 | 34(4) | 8(23.5)^{a} | 0^{a} | | | | | — |
| | with injection | | | | | | | | | |
| | 1-3 hours after | 136 | 45(5) | 32(71.1)^{b} | 7(15.6)^{b} | 3 | 2^{‡} | 2 | 0 | — |
| | injection | | | | | | | | | |
| | 3-6 hours after | 124 | 63(7) | 36(57.1)^{b} | 3(4.8)^{b} | 0 | 2^{‡‡} | 0 | 1^{‡‡{} | — |
| | injection | | | | | | | | | |
| B | 1-3 hour after | 1345 | 760(49) | — | — | — | — | — | — | 16(2.1) |
| | injection | | | | | | | | | |
| | 3-6 hour after | 62 | 40(5) | — | — | — | — | — | — | 1(2.5) |
| | injection | | | | | | | | | |
| C | 1-3 hour after | 458 | 298(18) | — | — | — | — | — | — | 6(2.0) |
| | injection | | | | | | | | | |
| D | 1-3 hour after | 603 | 287(18) | — | — | — | — | — | — | 8(2.8) |
| | injection | | | | | | | | | |

*Series A, Cesarean section were performed on 8.5 dpc or 11.5 dpc; Series B and C, Cesarean section were performed on 18.5-19.5 dpc. In Series A and B, each donor nucleus is from a B6D2F1 cumulus cell. In Series C, each donor nucleus is from a B6C3F1 cumulus cell. In Series D, each donor nucleus is from a B6C3F1 cloned mouse from Series C.
^{†}Superscripts ^{a} and ^{b} within the same column indicate significant difference between ^{a} and ^{b}: implantation(P < .005); fetal development(P < 0.05). The data were analyzed by Chi-square tests.
^{‡}Died 6-7 dpc;
^{‡‡}: Died 7-8 dpc;
^{‡‡‡}: Died 10 dpc

TABLE 3
Development of Enucleated Mouse Eggs Injected With Sertoli or Brain Cell Nuclei*

| | No. of | | | | | |
|---|---|---|---|---|---|---|
| | surviving | No.(%) of | Total no.(%) of | No. transferred | No.(%) of | |
| Cell type | oocytes | oocytes | morulae/blastocysts | embryos | Implantation | |
| injected | injected | activated | developed | (Recipient) | sites | Fetuses |
| Sertoli | 159 | 159(100) | 63(39.6)^{a} | 59(8) | 41(69.5) | 1(1.7) |
| Brain | 228 | 223(97.8) | 50(224)^{b} | 46(5) | 25(54.3) | 1(2.2) |

*All recipients were euthanized at 8.5 dpc. Superscripts ^{a} or ^{b} within the same column indicate a significant difference(P < 0.005) between and ^{b}.
^{†}Died about 6 to 7 dpc.
^{‡}Still alive at day 12.5 dpc.

TABLE 4
Development of Enucleated Mouse Eggs Injected with Various Types of Adult Somatic Cell Nuclei¹

| | | | No.(%) of oocytes | | No. of | | |
|---|---|---|---|---|---|---|---|
| | | No. of oocytes | | Developed to | transferred | No.(%) of | |
| Adult cell | Sex of | surviving after | | morulae/ | embryos | Implantation | |
| type | cell donor | nuclear transfer | Activated | blastocysts | (recipients) | sites | Fetuses |
| Thymus | Female | 176 | 168(95.5) | 5(3.1) | 0 | — | — |
| | Male | 96 | 58(60.4) | 0 | 0 | — | — |
| Spleen | Female | 80 | 49(61.3) | 11(22.4) | 11(2) | 10(90.9) | 2(18.2)* |
| | Male | 52 | 38(73.1) | 8(21.1) | 8(1) | 6(75) | 0 |
| Macrophage | Female | 308 | 187(60.7) | 58(31.0) | 52(5) | 26(50.0) | 4(7.7)* |
| | Male | 205 | 109(53.2) | 25(22.9) | 25(3) | 19(76.0) | 0 |

¹The number of implantation sits and fetuses were determined at 8.5 to 12.5 dpc.
*Died or stopped growing about 6 to 7 dpc.

TABLE 5
Full Term Development of Enucleated Mouse Oocytes Injected With Nuclei of Tail Fibroblasts of Adult Males:
Comparison of the Effect of Additional 3-5 Day Culture of Fibroblasts in Serum-Free
Medium After an Initial 5-7 Day Culture in Serum-Containing Medium

| Culture of fibroblasts | No. of | | | | | |
|---|---|---|---|---|---|---|
| in serum-free(−) or | enucleated | No. of injected oocytes | | No.(%) of oocytes | No. of transferred | No.(%) of |
| serum-containing(+) | oocytes | Surviving, | Activated | developed to morula/ | embryos | live off- |
| medium | injected | injected | (%) | blastocyst stage | (recipients) | spring^{†} |
| + | 467 | 414 | 327(78.9) | 162(49.5)* | 177(16) | 2(1.1) |
| − | 250 | 219 | 136(62.1) | 35(58.3)* | 97(9) | 1(1.0) |

*2-cell to blastocyst stage embryos were transferred into the oviducts or uteri of recipients.
^{†}No significant difference was observed in the success rate of obtaining live offspring between female and male clones. Data were analyzed by Chi-square tests.

TABLE 6
Full Term Development of Enucleated Mouse Qocytes After Injection of Cumulus Cell Nuclei
From Various Strains and Hybrids of the Mouse

| | | | | | | No. of | |
|---|---|---|---|---|---|---|---|
| | | No. of | No. of oocytes | | % activated oocytes | embryos | No.(%) of |
| Cumulus cell | Oocyte | enucleated | Surviving, | Activated | developed to morula/ | transferred | live |
| nucleus donor | recipient | oocytes | injected | (%) | blastocyst stage* | (recipients) | offspring^{§} |
| Inbred: | | | | | | | |
| C57BL/6 | B6D2F1 | 1098 | 1045 | 1006(96.3) | 23.8 | 413(24) | 0 |
| C3H/He | B6D2F1 | 322 | 305 | 297(97.4) | 48.4 | 200(16) | 0 |
| DBA/2 | B6D2F1 | 382 | 370 | 354(95.7) | 59.3 | 308(16) | 1(0.3) |
| DBA/2 | DBA/2 | 57 | 51 | 46(90.2) | —^{†} | 44(4) | 0 |
| | Subtotal | 1859 | 1771 | 1703(96.2) | | 965(60) | 1(0.1)^{a} |
| Hybrid: | | | | | | | |
| B6D2F1^{‡} | B6D2F1 | 1561 | 1522 | 1444(94.9) | 62.0 | 865(58) | 22(2.5) |
| B6C3F1^{‡} | B6D2F1 | 502 | 473 | 454(96.0) | 71.1 | 312(19) | 7(2.2) |
| B6D2F1 | B6C3F1 | 381 | 372 | 354(95.2) | 49.4 | 189(18) | 7(3.7) |
| B6C3F1 | B6C3F1 | 367 | 341 | 307(90.0) | 81.4 | 267(20) | 5(1.9) |
| | Subtotal | 2811 | 2708 | 2559(94.5) | | 1633(115) | 41(2.5)^{b} |

*These data were calculated when embryos were cultured to the blastocyst stage.
^{†}All 2-cell embryos were transferred to recipient females.
^{‡}These data include the data of Table 2, Series A and B for B6D2F1 mice, and Table 2, Series C for B6C3F1 mice.
^{§}Superscripts indicate a statistical significant difference between ^{a} and ^{b} (p < 0.005). Data were analyzed by Chi-square tests.

TABLE 7
Weight of Placenta of Cloned Mice at 19.5 Dpc

| | | Placenta | |
|---|---|---|---|
| Adult Somatic | | | Weight in grams*, |
| Cell Used for | | No. | mean ± standard deviation) |
| Cloning | Sex of Fetus | Examined | (range) |
| Cumulus | Female | 23 | 0.33^{a} ± 0.08 (0.21-0.61) |
| Fibroblast | Male | 3 | 0.34^{a} ± 0.07 (0.29-0.39) |
| — | Female | 10 | 0.12^{b} ± 0.02 (0.10-0.16) |
| | (non clone) | | |
| — | Male | 11 | 0.15^{b} ± 0.03 (0.10-0.18) |
| | (non clone) | | |

*Superscripts indicate statistical significant difference between a and b (p < 0.001). Data were analyzed by Student's t-tests.

We claim:

1. A method for cloning an animal comprising the steps of:
(a) collecting the nucleus of a fibroblast cell from an adult animal;
(b) inserting at least a portion of the fibroblast cell nucleus that includes the chromosomes into an enucleated oocyte to form a renucleated oocyte;
(c) allowing the renucleated oocyte to develop into an embryo; and
(d) allowing the embryo to develop into a live offspring.

2. The method of claim 1, wherein the fibroblast cell is a cultured cell.

3. The method of claim 1, wherein the fibroblast cell nucleus has 2n chromosomes.

4. The method of claim 1, wherein the fibroblast cell nucleus is 2C to 4C.

5. The method of claim 1, wherein the fibroblast cell nucleus is inserted into the cytoplasm of the enucleated oocyte.

6. The method of claim 5, wherein the inserting step is accomplished by microinjection.

7. The method of claim 6, wherein the microinjection is piezo electrically-actuated microinjection.

8. The method of claim 1, wherein the enucleated oocyte is arrested in the metaphase of the second meiotic division.

9. The method of claim 1, further comprising the step of activating the oocyte prior to, or during, or after the insertion of the fibroblast cell nucleus.

10. The method of claim 9, wherein the activation step takes place from zero to about six hours after the insertion of the fibroblast cell nucleus.

11. The method of claim 9, wherein the activation step takes place from about one to about three hours after the insertion of the fibroblast cell nucleus.

12. The method of claim 9, wherein the activation step comprises electroactivation, or exposure to a chemical activating agent.

13. The method of claim 12, wherein the chemical activating agent is selected from the group consisting of ethyl alcohol, sperm cytoplasmic factors, oocyte receptor ligand peptide mimetics, pharmacological stimulators of Ca²⁺ release, Ca²⁺ ionophores, strontium ions, modulators of phosphoprotein signaling, inhibitors of protein synthesis, and combinations thereof.

14. The method of claim 12, wherein the chemical activating agent is selected from the group consisting of caffeine, the Ca²⁺ ionophore A 23187, ethanol, 2-aminopurine, staurospurine, sphingosine, cyclohexamide, ionomycin, 6-dimethylaminopurine, and combinations thereof.

15. The method of claim 13, wherein the activating agent comprises Sr²⁺.

16. The method of claim 1, further comprising the step of disrupting microtubule and/or microfilament assembly in the oocyte for a time interval prior to or after insertion of the fibroblast cell nucleus.

17. The method of claim 16, wherein the time interval is zero to about 6 hours.

18. The method of claim 16, wherein the microtubule and/or microfilament assembly is disrupted by a selection from the group consisting of cytochalasin B, nocodazole, colchicine, and combinations thereof.

19. The method of claim 18, wherein the microtubule formation is disrupted by cytochalasin B.

20. The method of claim 1, further comprising the step of disrupting microfilaments in the oocyte for a time interval prior to or after insertion of the fibroblast cell nucleus.

21. The method of claim 20, wherein the time interval is from about zero to about 6 hours.

22. The method of claim 20, wherein the microfilaments are disrupted by cytochalasin D, jasplakinolide, latrunculin A, or combinations thereof.

23. The method of claim 1, wherein the step of allowing the embryo to develop into a live offspring further comprises the substep of transferring the embryo to a female surrogate recipient, wherein the embryo develops into a viable fetus.

24. The method of claim 1, wherein the inserting step further comprises inserting a reagent into the cytoplasm of said oocyte.

25. The method of claim 24, wherein the reagent is selected from the group consisting of an exogenous protein, a derivative of an exogenous protein, an antibody, a pharmacological agent, and combinations thereof.

26. The method of claim 24, wherein the inserting step further comprises inserting an exogenous nucleic acid or a derivative of an exogenous nucleic acid into the cytoplasm of said oocyte.

27. The method of claim 1, wherein the animal is male.

28. The method of claim 1, wherein the animal is female.

29. The method of claim 1, wherein the animal is selected from the group consisting of mammals, amphibians, fish and birds.

30. The method of claim 29, wherein the mammal is selected from the group consisting of primates, ovines, bovines, porcines, ursines, felines, canines, equines, and rodents.

31. The method of claim 30, wherein the mammal is a mouse.

32. An animal whose somatic and germline cells contain only the chromosomes derived from the nucleus of a fibroblast cell from an adult animal.

33. The animal of claim 32, wherein the animal is selected from mammals, amphibians, fish and birds.

34. The animal of claim 33, wherein the mammal is selected from the group consisting of primates, ovines, bovines, porcines, ursines, felines, canines, equines, and rodents.

35. The animal of claim 34, wherein the mammal is a mouse.

36. The animal of claim 32, wherein the animal is male.

37. The animal of claim 32, wherein the animal is female.

38. A method for modulating embryological development, comprising the steps of:
(a) combining a nucleus of a fibroblast cell from an adult animal with an enucleated oocyte to form a renucleated oocyte;
(b) inserting a reagent into the cytoplasm of the oocyte, prior to, during, or after the combining step; and
(c) allowing the reagent-treated renucleated oocyte to develop into an embryo.

39. The method of claim 38, wherein the reagent is selected from the group consisting of an exogenous protein, a derivative of an exogenous protein, an antibody, a pharmacological agent, an exogenous nucleic acid, a derivative of a exogenous nucleic acid, and combinations thereof.

40. The method of claim 38, wherein the inserting step comprises microinjection.

41. The method of claim 40, wherein the microinjection is piezo electrically-actuated microinjection.
